(19) United States
(12) Patent Application Publication
(10) Pub. No.: US 2005/0119640 A1
(43) Pub. Date: Jun. 2, 2005

(54) Surgical instrument for adhering to tissues

(75) Inventors: Leroy Sverduk, Lake Ariel, PA (US); Ranjana Sahai, El Cerrito, CA (US); Ronald S. Fearing, El Cerrito, CA (US)

Correspondence Address: MORRISON & FOERSTER LLP, 425 MARKET STREET, SAN FRANCISCO, CA, 94105-2482, US

(73) Assignee: The Regents of the University of California

(21) Appl. No.: 10/959,002

(22) Filed: Oct. 4, 2004

Related U.S. Application Data
(60) Provisional application No. 60/508,342, filed on Oct. 3, 2003.

(51) Int. Cl. A61B 17/00; A61B 18/04; A61B 19/00

(52) U.S. Cl. 606/1; 128/897; 128/898

(57) ABSTRACT

A surgical device capable of adhering to tissues is disclosed herein. The surgical device includes a micromechanical frame moveably linked to a plurality of micromechanical appendages. A plurality of nano-fibers that mimic adhesion of the Tokay Gecko are disposed at the terminus of each protrusion.

### RELATED APPLICATIONS

This application claims benefit of U.S. Provisional Application No. 60/508,342, filed Oct. 3, 2003, which is incorporated herein by reference in its entirety.

The U.S. Government has a paid-up license in the invention and the right in limited circumstances to require the patent owner to license others on reasonable terms as provided by the terms of Grant (Contract) No. IIS 0083472 and DMII 0115091 awarded by the National Science Foundation (NSF), and N00014-98-0671 awarded by ONR MURI.

### BACKGROUND

1. Field

This application relates generally to the fabrication and use of nano-scale adhesive structures disposed on surgical instruments.

2. Related Art

One of the most difficult challenges in surgical methods is carrying out surgery as minimally invasively as possible. The ability to guide surgical instruments remotely has dramatically improved surgical methods, frequently allowing surgery to be less invasive, to improve healing and recovery time of patients.

Many of the greatest challenges in minimally invasive surgery involve manipulating or treating moving tissues. Suturing a tissue, for example, requires precision and accuracy that is extremely difficult to control on a moving tissue, such as a beating heart. Conventionally, such surgical techniques require the tissue to suspend function. In the case of heart surgery, manipulation and treatment of a beating heart is accomplished by temporarily stopping the heart from beating or clamping in a local region.

In addition, conventional surgical devices use clamps, suction, and other similar devices to adhere to tissue. Such devices can damage the tissue. Moreover, such devices can interfere with the function or movement of the tissue.

### SUMMARY

In one embodiment, a surgical device is provided that is capable of adhering to tissues. The surgical device includes a micromechanical frame, a plurality of micromechanical appendages moveably linked to the micromechanical frame, and a plurality of nano-fibers disposed on the terminus of at least one micromechanical appendage. Each nano-fiber having a diameter between 50 nanometers and 2.0 microns and a length between 0.5 microns and 20 microns. Each nano-fiber is configured to provide an adhesive force on the surface of a tissue.

In another embodiment, a method of adhering the surgical device to a tissue is provided. The tissue is contacted with the terminus of one or more appendage. A plurality of nano-fibers is disposed on the terminus of one or more appendages to adhere to the tissue surface. The contacting step can include moving at least one appendage in the direction normal to the tissue surface, followed by moving at least one appendage in the lateral direction along the tissue surface, causing at least a portion of the nano-fibers to adhere to the surface. In another variation, the method can include detaching at least one appendage by increasing the angle of the terminus of at least one appendage relative to the tissue, to peel the appendage away from the tissue.

In another embodiment, a method of moving the surgical device along the surface of a tissue is provided. The method includes contacting the terminus of at least a portion of the plurality of appendages to the tissue surface, causing a portion of the nano-fibers disposed on the appendages to adhere to the tissue. At least one appendage is detached from the tissue by increasing the angle of the appendage relative to the tissue, breaking the adhesion of the nano-fibers with the tissue and peeling the appendage away from the tissue. The appendage is re-adhered to the tissue by contacting the appendage in the direction normal to the tissue surface, then moving the appendage in the lateral direction along the tissue surface, causing at least a portion of the plurality of nano-fibers disposed on the terminus of the appendage to adhere to the tissue.

In another embodiment, a method of making the surgical device is provided. A micromechanical frame is moveably linked to a plurality of micromechanical appendages. A plurality of nano-fibers is disposed on the terminus of at least one micromechanical appendage. Each nano-fiber has a diameter between 50 nanometers and 2.0 microns and a length between 0.5 microns and 20 microns. Each nano-fiber is configured to provide an adhesive force on the surface of a tissue.

### BRIEF DESCRIPTION OF THE DRAWINGS

FIG. 1 shows a perspective view of a micromechanical structure adhering to heart tissue, according to one embodiment of the application;

FIG. 2 shows a perspective view of a micromechanical frame and appendages incorporated into the micromechanical structure of FIG. 1;

FIG. 3 shows materials used in a piezoelectric actuator;

FIG. 4A shows an exemplary nano-fiber according to another embodiment;

FIG. 4B shows an exemplary nano-fiber according to another embodiment; and

FIG. 5 shows an exemplary nano-fiber contacting a rough surface.

### DETAILED DESCRIPTION

In order to provide a more thorough understanding of the present application, the following description sets forth numerous specific details, such as specific configurations, parameters, and the like. It should be recognized, however, that such description is not intended as a limitation on the scope of the present disclosure, but is intended to provide a better description of exemplary embodiments.

As further detailed herein, surgical instruments capable of adhering to organs and other tissues during minimally invasive surgery are provided. The surgical instruments include a micromechanical structure that adheres to tissues by micro-fibers via van der Waal's interactions. The micromechanical structure capable of adhering and moving along the surface of tissues, including moving tissues such as the heart muscle. Unlike conventional surgical robots, the surgical device can move on and in conjunction with the moving tissue such as a beating heart.

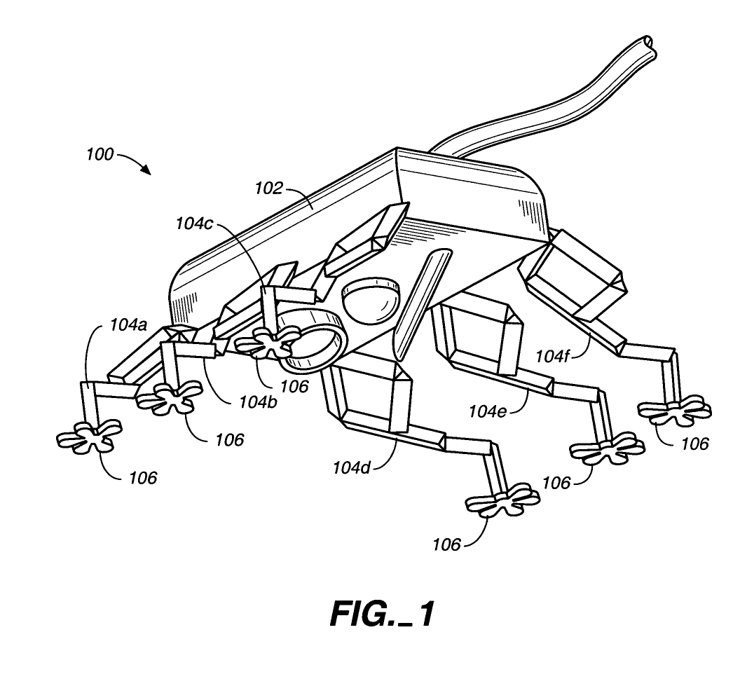

With reference to FIG. 1, according to one embodiment, surgical device 100 includes micromechanical frame 102 with a plurality of micromechanical appendages 104 moveably connected to micromechanical frame 102. Each micromechanical appendage 104a-f ends in foot section 106. A plurality of fabricated nano-fibers is disposed on the bottom section of each foot section 106. When introduced into the patient, the nano-fibers provide adhesion to patient tissue. Specifically, fabricated nano-fibers disposed on the bottom section of each foot section provide adhesion to the tissue. Adhesion of the surgical device is accomplished even when the tissue is moving.

Micromechanical Structures

The materials and components used to build the micromechanical structures disclosed herein are analogous to those described in U.S. Provisional Application No. 60/470,456 filed May 14, 2003, and U.S. Non-Provisional Application No. 10/830,374 filed Apr. 22, 2004, both of which are incorporated herein by reference in their entireties.

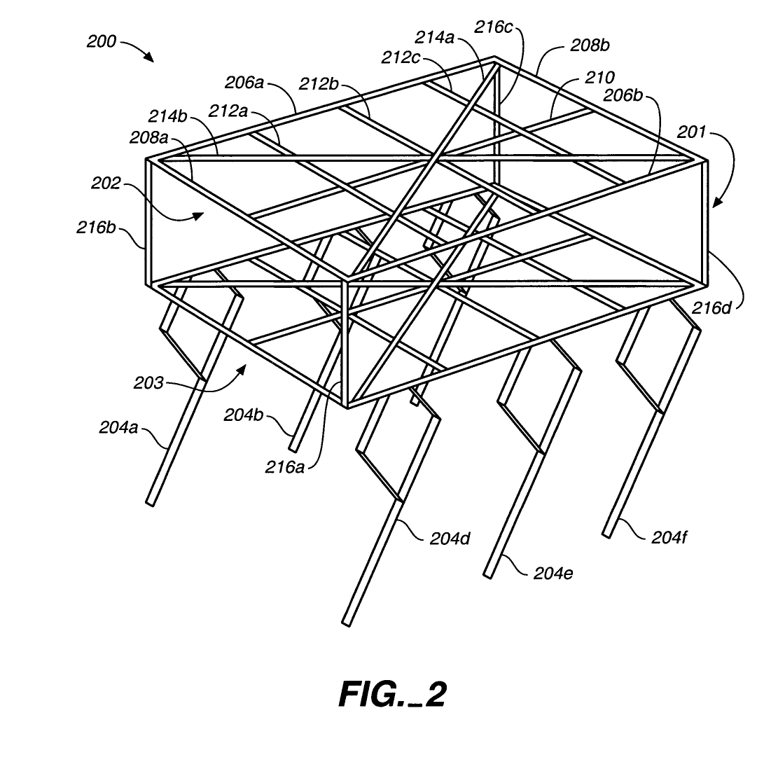

The micromechanical structure is designed to minimize weight while preserving structural integrity. With reference to FIG. 2, the micromechanical frame has a honeycomb structure. As used herein, honeycomb structures include any structure that resembles a honeycomb in structure or appearance. A honeycomb structure may include a cellular structural material or any structure that includes cavities like a honeycomb. Micromechanical structure 200 includes rectangular micromechanical frame 201, and six appendages 204a-f. Frame 201 has a top section 202 and bottom section 203 connected by support bars 216a-d. With reference to top section 202, the frame includes pair of side bars 206a and 206b, a pair of end bars 208a and 208b, longitudinal support beam 210, transverse bars 212a-c, and diagonal support beams 214a and 214b. Bottom section 203 is a mirror image of top section 202. Micromechanical frame 202 holds all actuators, drives, and motors of the micromechanical structure. In other embodiments, micromechanical frame 202 holds one or more of the actuators, drivers, or motors.

Components of micromechanical structures can be constructed using materials with a high stiffness to weight ratio. In such embodiments, components of composite micromechanical structures can be constructed using laser micromachining methods. In one embodiment, M60J carbon fiber reinforced epoxy was used. Experimentally, up to two cured plies can be cut simultaneously, or one uncured ply. To eliminate errors during construction of the cut laminae, all angles are controlled within the 2D CAD (Computer Aided Design) layout, and the plies are aligned optically under a microscope before cutting. Using uncured layers to construct the micromechanical structure has the great benefit of being able to lay-up the laminae for the links and a polymer for the joints at one time, and cure this laminate without the need of extra adhesive layers. Components of micromechanical structure can be constructed using only uncured laminae.

For frame and appendage components constructed from fiber-reinforced (e.g., carbon fiber-reinforced) beams/bars, uncured layers/materials are employed. Uncured layers/materials have the benefit of being able to lay-up the laminae for the links and the flexures a polymer for the joints at one time, and cure this laminate without the need of extra adhesive layers. In one exemplary embodiment of the present application, the frame and appendage components may possess the lamina parameter of the M60J composite.

Other materials, including but not limited to steel and silicon, may be used to construct the micromechanical structure. Configuration designed using composite materials provide stiffness with a minimum of added weight. Table 1 shows the lamina parameters of different materials. In other embodiments, the frame can be constructed from other materials, such as but not limited to steel or silicon. Non-composite materials, however, are not as strong and lightweight as composites such as M60J. Table 1 shows the lamina parameters of different materials.

TABLE 1

| | | M60J | | | |
|---|---|---|---|---|---|
| Param- | | Com- | | | |
| eter | Description | posite | Steel | Si | Units |
| E₁ | UHM longitudinal modulus | 350 | 193 | 190 | GPa |
| E₂ | UHM transverse modulus | 7 | 193 | 190 | GPa |
| v12 | UHM Poison's ratio | 0.33 | 0.3 | 0.27 | NA |
| G₁₂ | UHM shear modulus | 5 | 74 | 75 | GPa |
| t_{UHM} | UHM ply thickness | 25 | 12.5 | | microns |

The micromechanical structure disclosed herein is small enough to fit in a one and one half inch incision in an animal, such as a human. The incision can be, for example, between the ribs of a patient. In one embodiment, the mechanical frame is less than 4 centimeters in length and less than 4 centimeters in width. In various embodiments, the mechanical frame is less than 3 centimeters, 2.5 centimeters, 2 centimeters, 1.5 centimeters, or 1 centimeter in length. In various embodiments, the mechanical frame is less than 3 centimeters, 2.5 centimeters, 2 centimeters, 1.5 centimeters, or 1 centimeter in width. Those of skill in the art will recognize that any micromechanical structure capable of moving within a body during surgery can be substituted for the structure disclosed herein, provided that a plurality of nano-fibers are disposed on the appendages.

With further reference to FIG. 2, the micromechanical frame is moveably linked to one or more actuators (not shown), which are pivotably coupled to appendages 204a-f. Each actuator is coupled to electronics (not shown), creating a field across the actuator. In the embodiment of FIG. 2, a high mechanical power density required for movement of appendages 204a-f.

The one or more actuators can include piezoelectric materials and high modulus carbon fiber based passive layers. Under internal loading, the maximum achievable strain for an amorphous piezoceramic material (e.g. PZT-5H) is approximately 0.2%. Utilizing the thermal expansion properties of various composite materials for allows for extrinsically increasing the fracture toughness of these actuator materials. In addition, control of geometric factors, such as using a wedge planiform and extension, more uniformly distributes stress within the actuator, increasing peak strain energy. The strain energy density of the actuators is increased by a factor of 10 compared to commercial practice.

Each actuator may be constructed by laminating together a piezoelectric layer and an anisotropic passive layer in an ordered fashion and curing them together. The orientation, mechanical, and piezoelectric properties of the constituent materials are of importance for the performance of the actuators. With a mixture of piezoelectric materials and non-piezoelectric materials (e.g., anisotropic passive constituent layers(s)) within the actuators, either symmetric extension/contraction or uniform bending will occur when an electric field is applied to the piezoelectric material. Extension or contraction occurs when the piezoelectric materials are symmetric about the neutral axis while bending will occur when this symmetry does not exist. The anisotropic passive constituent layers produce a unidirectional composite that is capable of bending-twisting or extension-twisting coupling.

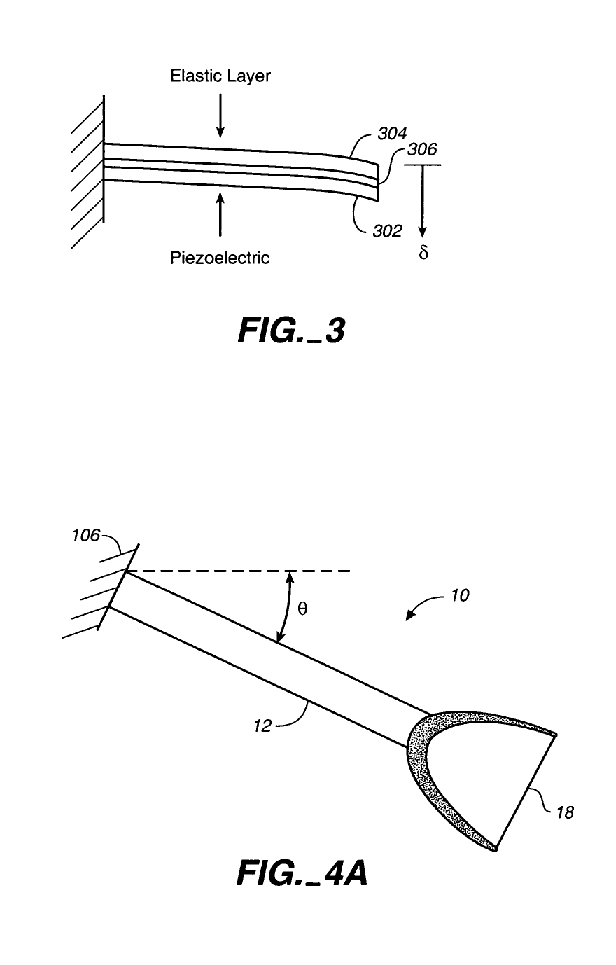

With reference to FIG. 3, in various embodiments, each actuator includes a piezoelectric layer 302, and a passive composite elastic layer 304 coupled to the piezoelectric layer 302 by a bonding layer 306. The bonding material for the bonding layer 306 may be any suitable bonding material, preferably a matrix epoxy. The bonding material for the bonding layer 306 may be purchased commercially from YLA Inc. of Benicia, Calif.

It will be recognized that any other actuation components known in the art, such as shape memory alloy, electrostrictive, electromagnetic, pneumatic, or optical, can be used in place of the actuators. The transmission components transmit power to the micromechanical appendages.

Micromechanical Appendages

With further reference to FIG. 1, a plurality micromechanical appendages is disposed on the micromechanical frame. The appendages may be designed to have one or more degrees of freedom.

To allow a micromechanical structure to maintain stability on a surface, three or more micromechanical appendages are disposed on the micromechanical frame. With further reference to the embodiment of FIG. 1, the one or more actuators disposed cause a force to be transmitted to appendages 104a-f. A foot section 106 is disposed at the terminus of each appendage. The appendages move, or “walk,” the micromechanical structure along a surface of the tissue.

In the present embodiment, each micromechanical appendage employs polyester flexures instead of revolute joints. The micromechanical appendages are constructed from layers of carbon fiber sheet with an intermediate polyester or other thin polymer layer sandwiched between these sheets. The polyester sheet has a thickness from 3 to 25 microns, and a flexure flexure length from 50 to 500 microns. The width of the carbon fiber link may be between 200 and 5000 micron. Alternatively, the micromechanical appendages are constructed from hollow stainless steel triangular beams that are used for the rigid elements of the structure. A folding fixture is constructed to bend stainless steel sheets and the determination of a folding angle sequence by static analysis using a compliant mechanism model. The appendages may be constructed from any material known in the art using any method, such as those described for the frame.

Those of skill in the art will recognize that the directional movement of the micromechanical structure can be controlled by changing the motion and direction of the micromechanical appendages. Appendages on different sides can detach, move forward, and re-attach to the tissue in an alternative fashion, producing a forward motion for the surgical device. Alternatively, appendages on a first side of the micromechanical structure can move farther than appendages on a second side of the structure, allowing the micromechanical structure to move forward and laterally relative to the tissue.

It will also be recognized that the appendages may be attached to the frame by any method known in the art.

Nano-Fibers

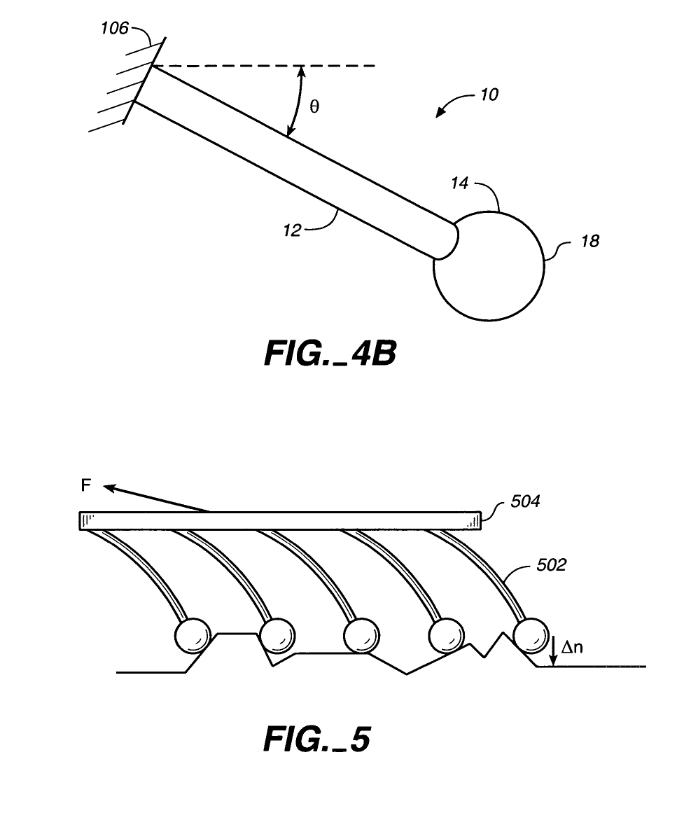

With further reference to FIG. 1, in one exemplary embodiment, the plurality of nano-fibers disposed on the bottom section of each foot section 106 mimic the adhesive properties of gecko feet. One embodiment of a nano-fiber is depicted in FIG. 4. Each nano-fiber 10 includes stalk 12 and terminal end 18. Terminal end 18 of nano-fiber 10 may be a paddle or flattened surface (FIG. 4A), a flattened segment of a sphere, a sphere, an end of a cylinder, or a curved segment of a sphere (FIG. 4B). Those of skill in the art will recognize that any type of structure may be placed at the terminus of a nano-fiber. Alternatively, the nano-fiber does not require an extended portion at the end of the nano-fiber.

In the present embodiment, nano-fiber 12 is between about 0.5 microns and 20 microns in length. The diameter of the nano-fiber stalk is between about 50 nanometers (nm) and 2.0 microns. As shown in FIGS. 4A and 4B, the nano-fibers or array of nano-fibers are supported at an oblique angle (neither perpendicular nor parallel) relative to foot section 106. This angle may be between about 15 and 75 degrees, and more preferably between about 30 degrees and 60 degrees. In the present embodiment, the angle is 30 degrees. In other embodiments, nano-fibers are not supported at an oblique angle, but at an angle perpendicular to foot section 106. With further reference to FIG. 1, the foot section surface 106 can be any material. In certain embodiments, nano-fibers can be made from such materials as polymers, for example, polyester, polyurethane and polyimide.

Each nano-fiber in FIG. 2, when in contact with contact surface 200, mimics the adhesive properties of nano-fibrous spatulae situated on setae of a Tokay Gecko. In certain embodiments, the average force provided at the contact surface by a single nano-fiber is between about 0.06 to 0.20 μN, or between about 60 and 200 nano-Newtons. In other embodiments, the average force provided at the contact surface by a single nano-fiber is between about 1.00 and 200 nano-Newtons. In other embodiments, the nano-fiber can provide a substantially normal adhesive force of between about 20 and 8,000 nano-Newtons. In still other embodiments, the nano-fiber can provide a substantially parallel adhesive force of between about 5 and 2,000 nano-Newtons.

An array of nano-fibers may be disposed at the terminus of one or more appendages, such as on the surface of one or more foot sections. In cases where only 10% of a 1000 nano-fiber array adheres to the contact surface with 2 μN adhesive force each, the array adheres to the contact surface with 200 μN adhesive force. Providing millions of such nano-fibers at the contact surface provides significantly greater adhesion.

Nano-fibers are also designed to be compatible with rough surfaces and smooth surfaces. Nano-fibers in contact with a rough surface are depicted in FIG. 5. By making the nano-fibers with a very high aspect ratio and very thin, they can adapt and adhere to rough surfaces when pressed against the surface. In addition, the nano-fibers adhere to both dry and wet surfaces. The well-known superhydrophobic nature of nano-structured fiber surfaces in particular, allows adhesion on wet surfaces such as those of tissues.

Nano-fibers achieve optimal adhesion when “pre-loaded” onto the tissue. As used herein, “pre-load” refers to providing a force on a nano-fiber normal to the contact surface, followed by a force parallel to the contact surface. With further reference to FIG. 5, when nano-fiber 502 first contacts a tissue surface it is pushed in a direction normal to the tissue surface. The foot section of the surgical instrument then moves in a direction lateral to the tissue, pulling the nano-fiber 502 laterally along the surface of the tissue. The small perpendicular preloading force in concert with a rearward displacement or-parallel preload provides significantly enhanced adherence to the tissue surface. In some embodiments the force of adhesion can increases by 20 to 60-fold, and adhesive force parallel to the surface increases linearly with the perpendicular preloading force. This initial perpendicular force need not be maintained during the subsequent pull. In addition, the “preloading” process is believed to increase the number of nano-fibers contacting the surface.

Nano-fibers on the surface of the surgical device can detached from the tissue by levering, or “peeling,” the nano-fiber away from the contact surface. The nano-fibers thus do not need to overcome the adhesive force between the nano-fiber and tissue to be removed from the tissue. This mechanism is described in U.S. patent application Ser. No. 10/197,763. In brief, nano-fibers are supported at angle relative to each foot section. When the foot section is rotated away form the tissue, the angle of incidence with respect to the tissue is increased. By changing the sliding direction (pushing or pulling the nano-fiber relative to the surface), a foot section with nano-fibers disposed thereon peels away from the tissue without explicitly pulling the terminus of the appendage away from the tissue. In one embodiment, a change in angle of adhesion of only 15% over a range of perpendicular forces results in detachment. In other embodiments, the detachment angle may be between about 25 degrees and 35 degrees.

The motion of each appendage can be designed to take advantage of pre-loading adhesion and peeling. To take advantage of the pre-loading capability of nano-fibers, the appendages can be configured to push normally, then laterally, along the tissue surface. When detaching from the tissue, the appendage can be designed to peel away from the tissue surface, causing the nano-fibers to release and the foot section to detach from the surface.

The micromechanical structure adheres to and follow the movement of the tissue without damaging the tissue or interfering with its movement. The nano-fibers do not damage or abrade the tissue to which they adhere. Moreover, adhesion to the tissue does not interfere with the movement of the tissue.

In another embodiment, nano-fibers may be built one upon the other to form a hierarchical nano-fiber geometry. Hierarchical nano-fibers may have a tree structure, where a large diameter base of perhaps six micron diameter branches into two or more nano-fibers of perhaps three micron diameter, which in turn each branch into two or more nano-fibers of lesser diameter, enhancing nano-fiber-to-contact surface compliance without a loss in effective nano-fiber stiffness. In this way, a material of higher stiffness, such as a high performance polymer or steel, can achieve an effective stiffness much less than that seen in an array of simple single diameter nano-fiber shafts, and thus heightened nano-fiber engagement, due to effectively more compliant nano-fibers.

By proper choice of nano-fiber length, angle, density and diameter, and substrate material, nano-fibers or arrays of nano-fibers can adhere to very rough surfaces. To avoid nano-fiber tangling, nano-fibers are optimally sufficiently stiff and separated while still dense sufficient to provide enough adhesion force. Arrays of nano-fibers can be constructed to prevent adhesion to each other. Further, nano-fibers can be constructed to have rough surface compatibility. The adhesive force of a nano-fiber depends upon its three-dimensional orientation (nano-fibers pointing toward or away from the surface) and the extent to which the nano-fiber is preloaded (pushed into and pulled along the surface) during initial contact. Further, a plurality of stalks can be disposed on the terminus of the appendages, and a plurality of nano-fibers can be disposed at the terminus of each stalk. A further discussion of all such design characteristics of nano-fibers is found in U.S. Pat. No. 6,737,160 and U.S. patent application Ser. No. 10/197,763, each of which is hereby incorporated by reference in its entirety.

The nano-fibers can be constructed by any material. In certain embodiments, the nano-fibers are produced by polyimide, polyester, and polydimethylsiloxane (PDMS), as described in U.S. patent application Ser. No. 10/197,763. The parameters for polyimide, polyester and polydimethylsiloxane (PDMS) rubber stalks are shown in Table 2. Note that the PDMS stalk has a length approximately less than or equal to its radius. This material provides adhesion to only perfectly planar contact surfaces.

TABLE 2

| Material | Pore Diameter (microns) | Thickness | Max. Temp | Pore Density |
|---|---|---|---|---|
| Alumina | UHM longitudinal modulus | 350 microns | 193 Celcius | 190 pores/sq. cm |
| Polycarbonate | UHM transverse modulus | 7 microns | 193 Celcius | 190 pores/sq. cm |

In other embodiments, the nano-fibers can be constructed from alumina having nanopore array. The nanopore array has 0.2 micron pore diameter. The alumina surface is 60 micron thick, and has 2×10⁹ pores/sq. cm. In other embodiments, the nano-fibers can be constructed from polycarbonate. The polycarbonate has a 0.2-10 micron pore diameter and is 7-20 microns thick. Its maximum temperature is 193 Celcius, and its pore density is generally between about 1×10⁴ and 2×10⁸ pores/sq.cm.

Surgical Tools

One or more surgical-tools can be disposed on the micromechanical structure. The surgical tool can be any tool or device known in the art. Examples of such surgical tools include endoscopic and laparoscopic tools used to move within or towards a target tissue (such as an organ) from a position outside the body. The tools include components that can be used to control the tools, as are well known in the art. It will be readily appreciated that wide variety of surgical tools and instruments include but are not limited to a Doppler flow meter, microphone, probe, retractor, dissector, stapler, clamp, grasper, needle driver, scissors or cutter, ablation or cauterizing elements, and surgical staplers, as are known in the art.

The surgical devices disclosed herein further include control and guidance electronics and components. The micromechanical structures disclosed herein can be coupled to other components.

Although the present application has been described with respect to certain embodiments, configurations, examples, and applications, it will be apparent to those skilled in the art that various modifications and changes may be made without departing from the application.

1. A surgical device adapted to adhere to tissues, comprising:
a micromechanical frame;
a plurality of micromechanical appendages moveably linked to the micromechanical frame; and
a plurality of nano-fibers disposed on the terminus of at least one micromechanical appendage, each nano-fiber having a diameter between 50 nanometers and 2.0 microns and a length between 0.5 microns and 20 microns, wherein each nano-fiber is adapted to provide an adhesive force on the surface of a tissue.

2. The surgical device of claim 1, wherein each nano-fiber is adapted to provide an adhesive force with the tissue of between 0.06 μN and 0.20 μN.

3. The surgical device of claim 1, wherein each nano-fiber is at an angle between 15 and 75 degrees relative to the foot section.

4. The surgical device of claim 3, wherein at least one said nano-fiber is at an angle between 30 and 60 degrees relative to the foot section.

5. The surgical device of claim 1, wherein a plurality of nano-fibers are disposed at the terminus of each said micromechanical appendage.

6. The surgical device of claim 1, wherein a plurality of stalk portions are disposed at the terminus of each appendage, wherein a portion of plurality of nano-fibers are disposed on the terminus of each stalk portion.

7. The surgical device of claim 5, wherein the terminus of each micromechanical appendage comprises a foot section.

8. The surgical device of claim 1, further comprising a surgical or diagnostic tool disposed on the micromechanical frame.

9. The surgical device of claim 8, wherein the tool is selected from the group consisting of a Doppler flow meter, microphone, probe, retractor, dissector, stapler, clamp, grasper, needle driver, scissors, cutter, ablation or cauterizing element, and surgical stapler.

10. The surgical device of claim 1, wherein the length of the micromechanical frame is less than 4 cm and the width of the micromechanical frame is less than 4 cm.

11. The surgical device of claim 1, wherein the micromechanical frame comprises carbon fiber material.

12. The surgical device of claim 1, further comprising control components to control the movement of the surgical device.

13. A method of adhering the surgical device to a tissue, comprising:
providing a surgical device having a micromechanical frame, a plurality of micromechanical appendages moveably linked to the micromechanical frame, and a plurality of nano-fibers 4 cm disposed on the terminus of at least one micromechanical appendage, each nano-fiber having a diameter between 50 nanometers and 2.0 microns and a length between 0.5 microns and 20 microns,
contacting the terminus of the at least one appendage to a tissue surface, causing at least a portion of the nano-fibers disposed on the at least one appendage adhere to the tissue, to adhere the surgical device to the tissue.

14. The method of claim 13, wherein the contacting step comprises
moving the terminus of the at least one appendage in the direction normal to the tissue; and
moving the at least one appendage in the lateral direction along the tissue surface, to cause one or more nano-fibers to adhere to the surface.

15. The method of claim 13, wherein the tissue is an organ.

16. The method of claim 14, wherein the organ is a heart.

17. The method of claim 15, wherein the heart is beating.

18. A method of moving a surgical device along the surface of a tissue, comprising:
providing a surgical device having a micromechanical frame, a plurality of micromechanical appendages moveably linked to the micromechanical frame, and a plurality of nano-fibers disposed on the terminus of at least one micromechanical appendage, each nano-fiber having a diameter between 50 nanometers and 2.0 microns and a length between 0.5 microns and 20 microns,
contacting the terminus of at least a portion of the appendages with the tissue surface, causing at least a portion of the nano-fibers disposed on the portion of appendages to adhere to the tissue,
detaching at least one appendage from the tissue by increasing the angle of the terminus of the at least one protrusion relative to the tissue, to break the adhesion of the one or more nano-fibers with the tissue and peeling the appendage away from the tissue;
re-adhering the at least one appendage to the tissue by contacting the at least one appendage in the direction normal to the tissue surface, then moving the at least one appendage in the lateral direction along the tissue surface, to cause at least a portion of the plurality of nano-fibers disposed on the terminus of the appendage to adhere to the tissue.

19. The method of claim 18, wherein the tissue is an organ.

20. The method of claim 19, wherein the organ is a heart.

21. The method of claim 20, wherein the heart is beating.

22. A method of making a surgical device, comprising:
providing a micromechanical frame;
moveably linking a plurality of micromechanical appendages to the micromechanical frame; and
disposing a plurality of nano-fibers on the terminus of at least one micromechanical appendage, each nano-fiber having a diameter between 50 nanometers and 2.0 microns and a length between 0.5 microns and 20 microns, wherein each nano-fiber capable of providing an adhesive force on the surface of a tissue.
